(19) United States
(12) Patent Application Publication
(10) Pub. No.: US 2016/0206548 A1
(43) Pub. Date: Jul. 21, 2016

(54) COCONUT OIL COMPOSITION AND METHOD OF USE

(71) Applicants: Eleanor Ismail, Cary, NC (US); Adam Ismail, Cary, NC (US); Srinivas Naga Chadaram, Durham, NC (US); Sharif Razzaque, Chapel Hill, NC (US)

(72) Inventors: Eleanor Ismail, Cary, NC (US); Adam Ismail, Cary, NC (US); Srinivas Naga Chadaram, Durham, NC (US); Sharif Razzaque, Chapel Hill, NC (US)

(21) Appl. No.: 15/001,827

(22) Filed: Jan. 20, 2016

Related U.S. Application Data
(60) Provisional application No. 62/105,458, filed on Jan. 20, 2015.

(51) Int. Cl. A61K 8/97; A61Q 19/00; A61K 8/02

(52) U.S. Cl. CPC A61K 8/97; A61K 8/0208; A61Q 19/007; A61K 2800/70

(57) ABSTRACT

A pre-moistened wipe is provided and comprises a substrate, and a composition comprising between about 80% to about 100% by volume of coconut oil, wherein the composition is releasably contacting the substrate. The substrate may be a fibrous material, including a nonwoven. In one aspect the composition comprises about 95% by volume coconut oil, about 1% by volume holy basil, about 1% by volume avocado oil, about 1% by volume aloe vera oil, about 1% by volume turmeric oil, and about 1% by volume neem oil. A method of delivering coconut oil to the skin is also provided and comprises a step of contacting skin with the wipe.

### CROSS-REFERENCES

This application is related to U.S. provisional application No. 62/105,458, filed Jan. 20, 2015, entitled “SYSTEMS, METHODS AND DEVICES FOR DELIVERY OF COCONUT OIL”, naming Eleanor Ismail, Adam Ismail, Srinivas Naga Chadaram and Sharif Razzaque as the inventors. The contents of the provisional application are incorporated herein by reference in their entirety, and the benefit of the filing date of the provisional application is hereby claimed for all purposes that are legally served by such claim for the benefit of the filing date.

### BACKGROUND

A composition including coconut oil is described for use on skin and, more particularly, the coconut oil composition is incorporated into a substrate to form a wet wipe for delivery to the skin.

Coconut oil has a pleasant odor and is a natural moisturizer and lubricant. There are numerous benefits to the application of coconut oil to, or in, the body. Coconut oil can reduce itching of an affected area of the skin. Coconut oil also deters the survival and growth of undesirable microorganisms, such as bacteria, viruses, and fungus, while restoring a healthy balance of microorganisms to the body. Further, coconut oil can be used in treating symptoms of yeast infections, in both men and women, on the surface of the skin and in body cavities. Examples include diaper rash, psoriasis, and vaginal yeast infections.

In its commonly available form, coconut oil can be inconvenient, difficult, and messy, to use. When used in a body cavity, coconut oil can leak out. This problem is compounded because the coconut oil must then be re-applied frequently.

Wet wipes may generally comprise a substrate pre-moistened with a fluidic composition for delivery to a surface. The substrate is commonly single layers of a nonwoven fibrous material, the fibrous material being cellulosic or polyolefin material. The composition may be aqueous, in which the components are freely soluble or stably dispersed within water. Wet wipes are useful for delivering functional materials to a surface. The composition may be suitable for use on a variety of surfaces, including skin. Wet wipes may provide skin benefits, such as conditioning or moisturizing the skin, or protection from or treatment of diaper rash. For wet wipes for use on skin, the composition may comprise surfactants, oils, skin care agents, pH buffers, solvents, preservatives, or other additives for cleaning or treating the skin.

For the foregoing reasons, there is a need for a new coconut oil composition and a convenient method and device for application of the coconut oil composition to the skin. The delivery method should facilitate the application and removal of coconut to any surface area of the skin. Ideally, a wet wipe is provided having a good delivery performance.

### SUMMARY

A pre-moistened wipe is provided and comprises a substrate, and a composition comprising between about 80% to about 100% by volume of coconut oil, wherein the composition is releasably contacting the substrate. The substrate may be a fibrous material, including a nonwoven. In one aspect the composition comprises about 95% by volume coconut oil, about 1% by volume holy basil, about 1% by volume avocado oil, about 1% by volume aloe vera oil, about 1% by volume turmeric oil, and about 1% by volume neem oil. A method of preparing a pre-moistened wipe is also provided, the method comprising the steps of providing a composition comprising between about 80% to about 100% by volume of coconut oil, and loading the composition onto a substrate to form a wet wipe. In addition, a method of delivering coconut oil to the skin is also provided and comprises a step of contacting skin with the wipe.

### DESCRIPTION

All of U.S. Pat. Nos. 9,119,779, 9,226,629, 9,125,528, 8,940,675, and 9,149,045, are incorporated herein by reference in their entirety.

Reference now will be made in detail to various embodiments of the composition and method, one or more examples of which are set forth below. Each example is provided by way of explanation of the composition and method, not limitation. In fact, it will be apparent to those skilled in the art that various modifications and variations may be made in the present composition and method without departing from the scope or spirit of the composition and method. For instance, features described as part of one embodiment may be used on another embodiment to yield a still further embodiment. Thus, it is intended that the present composition and method covers such modifications and variations as come within the scope of the appended claims and their equivalents. As used herein, percentages are given as the volume of the component to the total volume of the composition, unless otherwise indicated.

Values disclosed herein as ends of ranges are not to be understood as being strictly limited to the exact numerical values recited. Instead, unless otherwise specified, each numerical range is intended to mean both the recited values and any integers within the range. For example a range disclosed as “1 to 10” is intended to mean “1, 2, 3, 4, 5, 6, 7, 8, 9, 10.”

The term “coconut oil” as used herein is understood to mean virgin or refined, filtered or unfiltered, heat treated, bleached, deodorized, pasteurized or raw, expeller pressed, centrifuged, first-cold pressed or fractionated. Coconut oil may also be pure coconut oil, or mixed with other ingredients, including turmeric oil, holy basil oil, garlic, ginger, neem, tea-tree oil, aloe vera, eucalyptus oil, olive oil, avocado oil, xanthan gum, guar gum, thyme oil, xylitol, gelatin, agar-agar, or vitamins B,C,D E and K. Further, coconut oil may be whole coconut oil or extracts or components of whole coconut oil, including, for example, monolaurin, lauric acid, monocaprin, capric acid, monocaprylin, caprylin acid, monomyristin, or myristin acid, either alone or mixed with other components. Still further, coconut oil may be raw coconut oil or processed coconut or combined with agents to manipulate the melting temperature, shelf life, viscosity, scent, color, water solubility, and/or texture including, for example, hydrogenation, freeze drying, or agents such as propylene glycol, diazolidinyl urea, methylparaben, polysorbate, glycerol, gelatin, agar-agar, xanthan gum, guar gum or propylparaben.

The composition includes coconut oil. The composition may comprise from about 90% by volume to about 100% by volume of coconut oil. In one embodiment, the composition comprises about 98% coconut oil, about 1% avocado oil, and about 1% aloe vera oil. In another embodiment, the composition comprises about 97% coconut oil, about 1% holy basil, about 1% avocado oil, and about 1% aloe vera oil. In yet another embodiment, the composition comprises about 95% coconut oil, about 1% holy basil, about 1% avocado oil, about 1% aloe vera oil, about 1% turmeric oil, and about 1% neem oil. The composition may also include a carrier such as water. Additional ingredients may be added to the cleansing composition. The composition may include various optional ingredients, such as surfactants, oil materials, film-formers, preservatives, pH buffers, rheology modifiers, or other adjunct ingredients, and the like. Possible adjunct ingredients may be selected from a wide range of additional ingredients such as perfumes and fragrances, texturizers, colorants, soothing agents and medically active ingredients, such as healing actives and skin protectants.

Aspects of the present disclosure include a method of preparing a composition comprising coconut oil. The method comprises the steps of combining coconut oil and other optional ingredients, including holy basil, avocado oil, aloe vera oil, turmeric oil, and neem oil and mixing to form a cleansing composition. The composition may be prepared at a temperature in the range of 20-25.degree. C., according to the method.

The composition may be loaded onto a substrate to form a wet wipe and packaged such that the wipe is not accessible until the package is opened by a user. The components of the wipe may be formed from any of various suitable materials. Typically, however, the wipe includes a fibrous web that contains absorbent fibers. The fibers can be composed of cotton, nylon, paper, silk, felt, polyurethane, polypropylene, acrylic, nylon, Switchgrass, Lexan, Gore-Tex, polycarbonate, polystyrene, polypropylene silicon, hydrogel, BPA (corn plastic), fungus-derived plastics, ABS, wax, latex, rubber, polyester fibers, wood fiber papers, silk fibers, coconut husk, rice husk, hydrogel, hemp fibers, sugar cane fibers, bamboo fibers, or nylon fibers. It may be desired to have the materials be absorbent, biocompatible, compost-able or biodegradable.

Particularly well suited substrates include non-woven webs formed from synthetic or natural fibers. Preferably the non-woven webs are permeable to both air and liquids. The nonwoven material may comprise one or more layers of such fibrous assemblies, wherein each layer may include continuous fibers, coextruded fibers, non-continuous fibers and combinations thereof. In another embodiment, the wipe may comprise a laminate of a non-woven web and a liquid impermeable substrate. In addition, the wipe materials may be imparted with patterns or textures to facilitate the application of the composition. For example, when used to remove make-up the material of the wipe may include a texturized surface adapted to scrub or brush the skin of the user.

The wipe may assume a variety of shapes, including but not limited to, generally circular, oval, square, tubular, rectangular, or irregularly shaped. The shape of the wiping product make it particularly well suited for the application, such as, for example, removal of make-up and the like from the area around a user's eyes. In this embodiment, the shape of the wiping product would allow the user to easily access the recesses of the eye without incidental contact of the surrounding area or the eye itself. In this manner, the wipe would generally have a shape that is adapted to the morphology of the area of the user's skin. Regardless of shape, the total surface area of the wipe is from about 10 to about 20 cm.sup.2, more preferably from about 12 to about 15 cm.sup.2, although the surface area may vary based on intended application of the wipe.

During manufacture, the material of the wipe may be pre-formed and then dipped into melted coconut oil, or heated coconut oil may be dripped or sprayed onto the absorbent material. Alternatively, the coconut oil may be cooled or frozen and rolled or formed into a leaf, sheet, cake, tablet or rod shape. The wipe material may then be folded around the coconut oil and then sealed closed using adhesive, pressing, stamping, stitching, laser welding, etc. The resulting wipe may then be packaged inside a leak proof wrapper, film or envelope made of plastic, PRC (previously recycled, post consumer), BPA (corn plastic), coated paper, metallic foil, etc.

In use, the composition is applied to a wipe prior to use. Such wipes may be used to deliver the composition to a surface of skin of a user or another person. The wipe can be used to wipe, clean or apply the composition onto skin of adults, infants, and children, or animals, including around the mouth, eyes, nostrils, ear canal, labia, vagina, anus, rectum, etc. The wipe could also be rolled, folded, wadded or pressed, and then inserted into a body cavity, such as the nose, vagina or rectum. In one embodiment, the wipe can be provided pre-rolled, folded, wadded or pressed, to make the wipe more convenient for insertion into a body cavity. If the user prefers to use this wipe for another purpose, the user can first unroll or unfold the wipe. The wipe may provide an increased surface area to facilitate contact of the composition with the surface. In addition, the wipe may also serve other purposes, such as providing water absorption, barrier properties, etc. The wipe may also eliminate microorganisms through frictional forces imparted to the surface.

A suitable package for storing and dispensing the wipe may include a non-rigid container having sides with a top end portion and a bottom end portion where the sides and top and bottom end portions define a cavity within the container. The cavity is configured to provide a storage portion for the wipe. The top end portion can include a re-sealable mechanism.

A single wipe wetted with the present composition may be provided to consumers in quantities of one in a package of convenient size, for example, no greater than a dimensional size of 3.5 inches long or wide by 4 inches long or wide by 1 inch deep. A plurality of wipes wetted with the present composition may be provided to consumers in a stack of wipes, i.e., two or more wipes stacked on top of one another. The plurality of wipes can be arranged in a package or dispenser in any manner which provides convenient and reliable one at a time dispensing. The axis of adjacent wipes may be offset from one another, which portions of the adjacent wipes to remain accessible to a user and improves dispensing. Each individual wipe may be arranged in a folded configuration and stacked one on top of the other to provide a stack of wet wipes. Such folded configurations are well known to those skilled in the art and include c-folded, z-folded, quarter-folded configurations and so forth.

While the composition and the wipe are particularly well suited for skin, such as the removal of makeup around the eyes, this is but one suggested use of the wipe. The wipe may be used in various applications. For example, the wipe may be used to clean or treat parts of the body such as the eyes, the ears, the nose, and the like. The wipe also may be used to apply the composition to any part of the body, any other object or cavity. It should be appreciated however, that the aforementioned options for the use of the wipe is not intended as a limitation. It is also understood that the composition may be used with various substrates, including tissues, paper towel, toilet paper, and the like. The substrates may be directly loaded with the composition, or the composition may be applied to the substrate at the time of use in the form of a liquid or spray. The substrates may then be used to clean various other surfaces of the skin.

What is claimed is:

1. A pre-moistened wipe comprising:
a substrate, and
a composition comprising between about 80% to about 100% by volume of coconut oil,
wherein the composition is releasably contacting the substrate

2. The wipe as recited in claim 1, wherein the substrate comprises fibrous material.

3. The wipe as recited in claim 2, wherein the fibrous material is absorbent fibers.

4. The wipe as recited in claim 2, wherein the fibrous material is synthetic thermoplastic fibers.

5. The wipe as recited in claim 1, wherein the substrate is textured.

6. The wipe as recited in claim 2, wherein the substrate comprises a nonwoven.

7. The wipe as recited in claim 1, wherein the composition is in the form of a liquid.

8. The wipe as recited in claim 1, wherein the composition comprises about 95% by volume coconut oil, about 1% by volume holy basil, about 1% by volume avocado oil, about 1% by volume aloe vera oil, about 1% by volume turmeric oil, and about 1% by volume neem oil.

9. The wipe as recited in claim 1, wherein the composition comprises about 97% by volume coconut oil, about 1% by volume holy basil, about 1% by volume avocado oil, and about 1% by volume aloe vera oil.

10. The wipe as recited in claim 1, wherein the composition comprises about 98% by volume coconut oil, about 1% by volume avocado oil, and about 1% by volume aloe vera oil.

11. The wipe as recited in claim 1, wherein the composition comprises about 100% by volume coconut oil.

12. The wipe as recited in claim 1, wherein the composition comprises from about 80% to about 100% by weight or volume of coconut oil, and up to about 20% by weight or volume of other essential oils or natural ingredients selected from group of garlic, turmeric, neem, xylitol, stevia leaf, tea tree, vitamin E, K, monolaurin, gelatin, hibiscus, lemon, lavender, rose, jasmine, clove, cinnamon, cumin, or a combination thereof of oils and ingredients

13. A method of preparing a pre-moistened wipe, the method comprising the steps of:
providing a composition comprising between about 80% to about 100% by volume of coconut oil, and
loading the composition onto a substrate to form a wet wipe.

14. The method of preparing a wipe as recited in claim 13, wherein the composition is heated below 25 degree Celsius, and the loading step comprises dripping or spraying the composition onto the substrate.

15. The method of preparing a wipe as recited in claim 13, wherein the composition is heated below 23 degree Celsius, and the loading step comprises forming the composition into a leaf, sheet, or cake, and wrapping the composition in the substrate.

16. A method of delivering coconut oil to the skin comprising a step of contacting skin with the wipe of claim 1.
